(19) United States
(12) Patent Application Publication
(10) Pub. No.: US 2021/0023365 A1
(43) Pub. Date: Jan. 28, 2021

(54) SLEEP APNEA TREATMENT DEVICE

(71) Applicants: Tsung-Li LO, (US); Te-Hao LU, (US)

(72) Inventors: Tsung-Li LO, Taipei City (TW); Te-Hao LU, Hsinchu City (TW); Yi-Ting LU, Hsinchu City (TW)

(21) Appl. No.: 16/937,537

(22) Filed: Jul. 23, 2020

(30) Foreign Application Priority Data
Jul. 24, 2019 (TW) 108126228

(51) Int. Cl. A61N 1/36; A61N 1/378

(52) U.S. Cl. CPC A61N 1/3601; A61N 1/3787; A61N 1/36003

(57) ABSTRACT

A sleep apnea treatment device includes a buried breathing stimulation capsule for embedding in a user's lower jaw and the buried breathing stimulation capsule includes a power receiving coil and an output electrode. The power receiving coil is used for receiving a power required for the buried breathing stimulation capsule, and the output electrode is electrically connected to the power receiving coil, and emits a predetermined voltage to a genioglossus muscle of the user to cause contraction of the genioglossus muscle.

### RELATED APPLICATIONS

This application claims priority to Taiwan Application Serial Number 108126228, filed Jul. 24, 2019, which is herein incorporated by reference.

### TECHNICAL FIELD

The present disclosure generally relates to a sleep apnea treatment device. More particularly, the present disclosure relates to a wireless sleep apnea treatment device.

### BACKGROUND

With the advancement of science and technology and the advancement of medical technology, the life expectancy of human beings has gradually increased. A normal respiratory rate for healthy adults is between 12 and 20 breaths per minute. Obstructive sleep apnea syndrome (OSAS) is characterized by episodes of partial or complete obstruction of the upper respiratory tract during sleep, thereby interrupting or reducing the flowing rate of air. Some minor patients will have symptoms such as headache, memory loss, night sweats, and sleeplessness, and some severe patients may cause high blood pressure, heart failure, apoplexy and even sudden death.

In general, obstructive sleep apnea syndrome is the obstruction of the respiratory tract during sleep due to the loss of muscle tension. The muscles that open or close the respiratory tract are almost attached to the upper and lower jaw bones.

Currently, part of the treatment used to treat the obstructive sleep apnea syndrome is surgery and another part is to wear a respirator to improve the illness symptom. However, the traditional treatment is expensive and may not cure the obstructive sleep apnea syndrome.

There is a need to provide a sleep apnea treatment device that is convenient to wear and does not affect the patient's daily life so as to effectively improve the breathing condition of the patient and the life quality of the patient.

### SUMMARY

One objective of the embodiments of the present invention is to provide a sleep apnea treatment device, thereby effectively treating the symptoms of the respiratory arrest.

To achieve these and other advantages and in accordance with the objective of the embodiments of the present invention, as the embodiment broadly describes herein, the embodiments of the present invention provides a sleep apnea treatment device having a buried breathing stimulation capsule configured to be fixed in a lower jaw of a user. The buried breathing stimulation capsule includes a power receiving coil and an output electrode. The power receiving coil receives an electrical power for the buried breathing stimulation capsule, and the output electrode is electrically connected to the power receiving coil to emit a predetermined voltage to stimulate a tongue muscle of the lower jaw of the user so as to cause a contraction of the tongue muscle.

In some embodiments, the buried breathing stimulation capsule further includes a buck-boost circuit disposed between the power receiving coil and the output electrode to provide a desired voltage for the output electrode.

In some embodiments, the tongue muscle includes a genioglossus muscle located under a tongue and attached to an inner surface of a mandibular bone.

In some embodiments, the sleep apnea treatment device further includes a wireless power supply module to provide the electrical power for the buried breathing stimulation capsule by wireless induction.

In some embodiments, the wireless power supply module includes a power output coil, an inverter and a controller. The power output coil wirelessly induces the power receiving coil to provide the electrical power for the buried breathing stimulation capsule. The inverter is connected to the power output coil to adjust an alternating current power for the power output coil. In addition, the controller is connected to the inverter to control the inverter to output the alternating current power.

In some embodiments, the controller includes a logic controller.

In some embodiments, the controller includes an AC-DC converter.

In some embodiments, the wireless power supply module further includes a signal interface connected to the controller to input breathing condition signals of the user.

In some embodiments, the wireless power supply module further includes a human machine interface connected to the controller for the user to control the sleep apnea treatment device.

In some embodiments, the wireless power supply module further includes a power input unit connected to the controller to provide power for the wireless power supply module.

Hence, the sleep apnea treatment device can facilitate the patient to install a buried breathing stimulation capsule in the lower jaw thereof, and use a wireless power supply module to provide a desired electrical power for the buried breathing stimulation capsule during sleep. The wireless power supply module provides inductive electrical power for the buried breathing stimulation capsule to stimulate the tongue muscle of the patient when the patient temporarily stops breathing, so that the respiratory tract is unobstructed. During general daytime activities, the buried breathing stimulation capsule is very small and does not need store the electrical power in the capsule, so that the buried breathing stimulation capsule does not affect the patient's daily work and can be worn for a long time.

### BRIEF DESCRIPTION OF THE DRAWINGS

The foregoing aspects and many of the attendant advantages of this invention will be more readily appreciated as the same becomes better understood by reference to the following detailed description, when taken in conjunction with the accompanying drawings, wherein:

FIG. 1 illustrates a schematic view showing a sleep apnea treatment device according to one embodiment of the present invention.

FIG. 2 illustrates a schematic side view showing a sleep apnea treatment device of FIG. 1 installed in a lower jaw of a user.

FIG. 3 illustrates a schematic top view showing a position of the sleep apnea treatment device of FIG. 2 installed in the lower jaw of the user.

### DETAILED DESCRIPTION OF THE PREFERRED EMBODIMENT

The following description is of the best presently contemplated mode of carrying out the present disclosure. This description is not to be taken in a limiting sense but is made merely for the purpose of describing the general principles of the invention. The scope of the invention should be determined by referencing the appended claims.

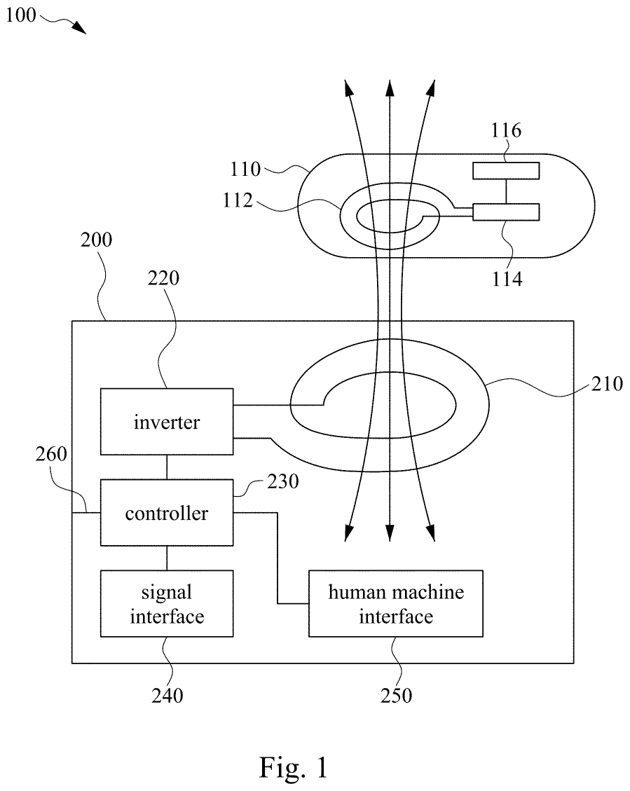

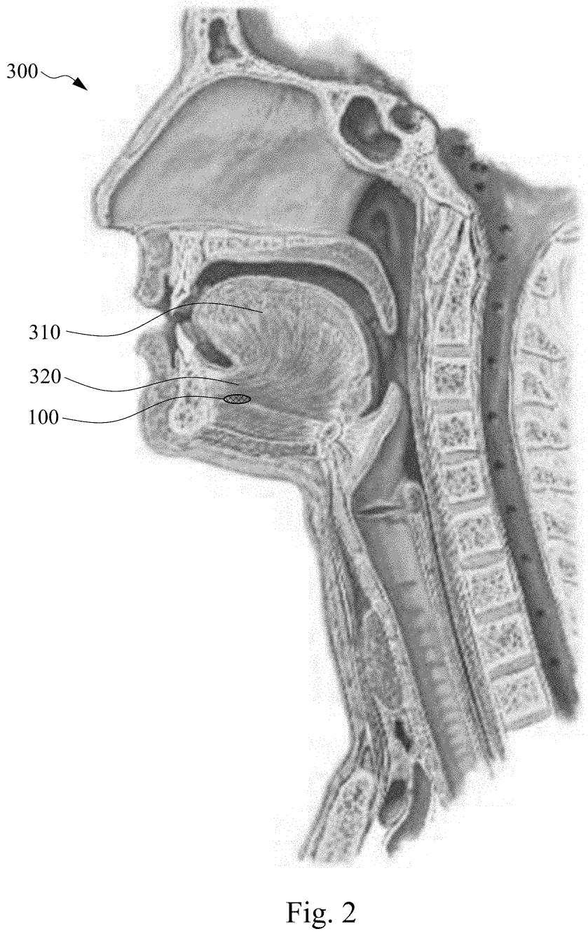

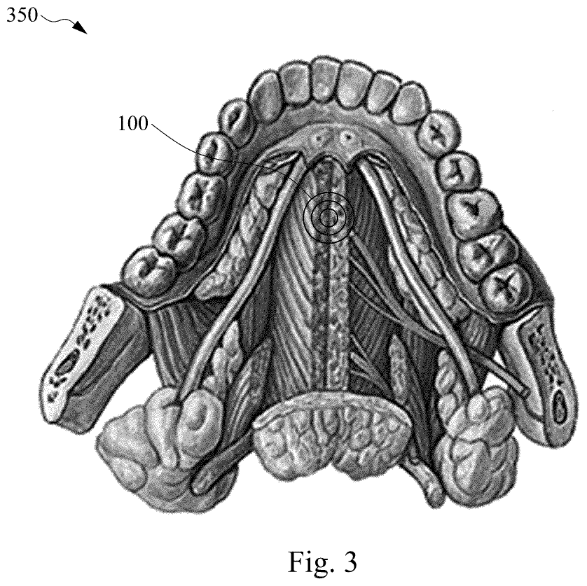

Refer to FIGS. 1-3. FIG. 1 illustrates a schematic view showing a sleep apnea treatment device according to one embodiment of the present invention, FIG. 2 and FIG. 3 illustrate the sleep apnea treatment device installed in a lower jaw of a user.

The sleep apnea treatment device 100 includes a buried breathing stimulation capsule 110, and the buried breathing stimulation capsule 110 includes a power receiving coil 112, a buck-boost circuit 114 and an output electrode 116.

The power receiving coil 112 is utilized to receive the electrical power for operating the buried breathing stimulation capsule 110. In some embodiments, the power receiving coil 112 is a wireless power receiving coil 112 to receive the electrical power from an exterior power output coil by wireless induction. Therefore, the buried breathing stimulation capsule 110 can be embedded and operated in the human body without a battery equipped in the buried breathing stimulation capsule 110 or an exterior power cord directly connected to the buried breathing stimulation capsule 110.

The output electrode 116 can emit a predetermined voltage, i.e. 6 volts to 20 volts, to stimulate the tongue muscle, i.e. genioglossus muscle 320, to cause contraction of the genioglossus muscle 320. The buck-boost circuit 114 is disposed between the power receiving coil 112 and the output electrode 116 to provide the desired voltage for the output electrode 116.

Simultaneously referring to FIGS. 2 and 3, the buried breathing stimulation capsule 110 can be conveniently installed in the human head 300 and located under the tongue 310, i.e. the position of the user's lower jaw 350 as illustrated in FIG. 2 and FIG. 3.

When a doctor diagnoses the patient having an apnea symptom, the patient does not need to perform operations as a traditional patient as well as the patient does not need to wear an inconvenient respirator and other devices. The patient only needs local anesthesia at the bottom of lower jaw 350, and a small incision is made in the mucosa of the lower jaw 350, and the buried breathing stimulation capsule 110 is installed therein to stimulate the genioglossus muscle 320 attached to the inner surface of the lower mandibular bone. When the genioglossus muscle 320 is stimulated by the output electrode 116, the voltage can stimulate the genioglossus muscle 320 to cause contraction of the genioglossus muscle 320 to expand the upper respiratory tract. When the genioglossus muscle 320 is stimulated to cause contraction, the genioglossus muscle 320 may increase the tension of the upper respiratory tract muscles to strengthen the respiratory tract strengthens and increase the space of the upper respiratory tract to maintain airflow flowing freely, and reduces the airflow resistance in the upper respiratory tract, so that the patient can breathe normally and the symptoms of respiratory arrest can be effectively treated.

The volume of the buried breathing stimulation capsule 110 is very small, and therefore the buried breathing stimulation capsule 110 can be conveniently embedded in the lower jaw 350. In addition, the buried breathing stimulation capsule 110 can be operated without a battery storing energy for long term working, so that the buried breathing stimulation capsule 110 can be embedded in the lower jaw 350 for a long time without having to worry about the problems of the battery leakage, the battery exhaustion and the weight of the device.

In some embodiments, the sleep apnea treatment device 100 further includes a wireless power supply module 200 to provide the electrical power for the buried breathing stimulation capsule 110 through the wireless induction.

The wireless power supply module 200 includes a power output coil 210, an inverter 220, a controller 230 and a power input unit 260. The power input unit 260 is connected to the controller 230 to provide power for the wireless power supply module 200. The power input unit 260 can be an external power input terminal or a battery.

The power output coil 210 is electrically connected to the controller 230 to provide the electrical power to the buried breathing stimulation capsule 110 through the power receiving coil 112 of the buried breathing stimulation capsule 110.

The inverter 220 is connected to the power output coil 210 and the controller 230 to adjust an alternative current power for the power output coil 210. The inverter 220 can use solid-state electronic components to adjust the voltage and frequency. The inverter 220 can receive direct current (DC) power to convert the same to alternating current (AC) power with a required frequency, or convert AC power to AC power with the required frequency without departing from the spirit and scope of the invention.

The controller 230 is connected to one end to the power input unit 260 and is connected to the inverter 220 to provide the required power for the inverter 220 and controls the inverter 220 to output AC power with the required frequency.

In some embodiments, the controller 230 includes a logic controller, for example, a microcontroller (MCU) or the like.

In some embodiments, the controller 230 further includes an AC-DC converter to convert the grid power to a required DC power and supply to the inverter 220.

In some embodiments, the wireless power supply module 200 further includes a signal interface 240 connected to the controller 230, and receives breathing condition signals of the users, e.g. patients, for the controller 230 from external sensors or internal sensors. When the controller 230 determines that the patient has stopped breathing too long, i.e. 8-10 seconds or more, the controller 230 transmits the electrical power to the inverter 220, and then the inverter 220 forms the required AC power to the power output coil 210 to induce the power receiving coil 112 of the buried breathing stimulation capsule 110. In addition, the electrical power induced by the power receiving coil 112 is then transmitted to the buck-boost circuit 114 to boost the voltage and transmit to the output electrode 116 to stimulate the genioglossus muscle 320 to open the respiratory tract.

Therefore, the patient only needs to stick or place the power output coil 210 of the wireless power supply module 200 near the lower jaw 350, and then the power output coil 210 can induce the power receiving coil 112 of the buried breathing stimulation capsule 110. In some embodiments, the power output coil 210 may be disposed on the exterior shell of the wireless power supply module 200 so as to easily induce the power receiving coil 112. In other embodiments, the power output coil 210 may be disposed inside the shell of the wireless power supply module 200 and the wireless power supply module 200 is disposed close to the power receiving coil 112 so as to supply the wireless power, which does not depart from the spirit and scope of the present invention.

In some embodiments, the wireless power supply module 200 further includes a human machine interface 250, which is connected to the controller 230, so that the patient can use the human machine interface 250 to control the sleep apnea treatment device 100, and store relevant data for the doctor.

Accordingly, the sleep apnea treatment device can facilitate the patient to install a buried breathing stimulation capsule in the lower jaw thereof, and use a wireless power supply module to provide a desired electrical power for the buried breathing stimulation capsule during sleep. The wireless power supply module provides inductive electrical power for the buried breathing stimulation capsule to stimulate the tongue muscle of the patient when the patient temporarily stops breathing, so that the respiratory tract is unobstructed. During general daytime activities, the buried breathing stimulation capsule is very small and does not need store the electrical power in the capsule, so the buried breathing stimulation capsule does not affect the patient's daily work and can be worn for a long time. In addition, in some embodiments, the buried breathing stimulation capsule can also be equipped with a small battery and a control module installed in the buried breathing stimulation capsule to independently operate, without departing from the spirit and scope of the present invention.

As is understood by a person skilled in the art, the foregoing preferred embodiments of the present invention are illustrative of the present invention rather than limiting of the present invention. It is intended that various modifications and similar arrangements be included within the spirit and scope of the appended claims, the scope of which should be accorded the broadest interpretation so as to encompass all such modifications and similar structures.

What is claimed is:

1. A sleep apnea treatment device having a buried breathing stimulation capsule configured to be fixed in a lower jaw of a user, the buried breathing stimulation capsule comprising:
a power receiving coil receiving an electrical power for the buried breathing stimulation capsule; and
an output electrode electrically connected to the power receiving coil to emit a predetermined voltage to stimulate a tongue muscle of the lower jaw of the user so as to cause a contraction of the tongue muscle.

2. The sleep apnea treatment device of claim 1, wherein the buried breathing stimulation capsule further comprises a buck-boost circuit disposed between the power receiving coil and the output electrode to provide a desired voltage for the output electrode.

3. The sleep apnea treatment device of claim 1, wherein the tongue muscle comprises a genioglossus muscle located under a tongue and attached to an inner surface of a mandibular bone.

4. The sleep apnea treatment device of claim 1, further comprising a wireless power supply module to provide the electrical power for the buried breathing stimulation capsule by wireless induction.

5. The sleep apnea treatment device of claim 4, wherein the wireless power supply module comprises:
a power output coil wirelessly inducing the power receiving coil to provide the electrical power for the buried breathing stimulation capsule;
an inverter connected to the power output coil to adjust an alternating current power for the power output coil; and
a controller connected to the inverter to control the inverter to output the alternating current power.

6. The sleep apnea treatment device of claim 5, wherein the controller comprises a logic controller.

7. The sleep apnea treatment device of claim 6, wherein the controller further comprises an AC-DC converter.

8. The sleep apnea treatment device of claim 5, wherein the wireless power supply module further comprises a signal interface connected to the controller to input breathing condition signals of the user.

9. The sleep apnea treatment device of claim 8, wherein the wireless power supply module further comprises a human machine interface connected to the controller for the user to control the sleep apnea treatment device.

10. The sleep apnea treatment device of claim 9, wherein the wireless power supply module further comprises a power input unit connected to the controller to provide power for the wireless power supply module.
